(19) United States
(12) Patent Application Publication
(10) Pub. No.: US 2016/0338598 A1
(43) Pub. Date: Nov. 24, 2016

(54) BIOLOGICAL INFORMATION MEASUREMENT APPARATUS

(71) Applicant: SEIKO EPSON CORPORATION, Tokyo (JP)

(72) Inventor: Kazuyoshi KEGASAWA, Hara-mura (JP)

(73) Assignee: SEIKO EPSON CORPORATION, Tokyo (JP)

(21) Appl. No.: 15/161,150

(22) Filed: May 20, 2016

(30) Foreign Application Priority Data
May 22, 2015 (JP) 2015-104279

(51) Int. Cl. A61B 5/0205; A61B 5/0408; A61B 5/04; A61B 5/00

(52) U.S. Cl. CPC A61B 5/0205; A61B 5/681; A61B 5/04085; A61B 5/04012

(57) ABSTRACT

A biological information measurement apparatus including a case section including a biological information detector that detects biological information on a user, a band that attaches the case section to the user, a first electrode provided on a contact surface of the case section or the band that comes into contact with the user's body, a pinched section provided as part of the band and pinched by the user with fingers, a second electrode provided on the pinched section, and an electrocardiogram detector that uses the first electrode and the second electrode to detect the user's electrocardiogram.

### CROSS-REFERENCES TO RELATED APPLICATIONS

This application claims priority to Japanese Patent Application No. 2015-104279, filed May 22, 2015, the entirety of which is herein incorporated by reference.

### BACKGROUND

1. Technical Field

The present invention relates to a biological information measurement apparatus.

2. Related Art

As a biological information measurement apparatus, there has been a proposed apparatus of related art in which a plurality of electrodes are provided and electrocardiographic information, pulse wave information, and other types of biological information are measured through the electrodes. Further, in recent years, as people are increasingly health cautious, a compact biological information measurement apparatus capable of readily measuring biological information at home and making good use of the information for daily health control has been developed. For example, JP-A-2003-325468 discloses, as a portable biological information measurement apparatus, a wristwatch-shaped biological information measurement apparatus having a band section that allows a case section to be attached to a user's wrist.

The biological information measurement apparatus disclosed in JP-A-2003-325468 includes a first electrode and a second electrode caused to come into contact with two different points on the user's body, a biological information detector (biological information detector) that uses the first and second electrodes to detect biological information, such as the user's electrocardiogram, a case section (main body) that accommodates the biological information detector, and a band section (attachment section) that attaches the case section to the user's arm (wrist). The first electrode, which is one of the pair of electrodes, is provided on the case section or the band section, specifically, on a surface thereof that comes into contact with the user's body (skin) in a state in which the case section is attached via the band section. On the other hand, the second electrode is provided on a surface different from the surface on which the first electrode is provided, for example, on the side surface of the case section. That is, in the state in which the biological information measurement apparatus is attached to the user's arm, the first electrode keeps in contact with the user's body, and touching the second electrode with a finger of the hand of the arm different from the arm to which the biological information measurement apparatus is attached allows detection of biological information, such as the user's electrocardiogram, by using the first and second electrodes.

However, the biological information measurement apparatus described in JP-A-2003-325468, which employs a method that causes the user to press a finger against the second electrode to measure an electrocardiogram, it is difficult to maintain the state in which the second electrode is in contact with the user's finger (skin) in a stable manner for a predetermined period required for the measurement. Since the electrocardiogram measurement, among a variety of types of biological information measurement, requires signal components over a particularly wide frequency range, an unstable state in which the measurement electrodes are in contact with the user's body may undesirably disturb the resultant electrocardiographic waveform due to influence of disturbing factors present in the electrocardiographic frequency band and surrounding bands.

### SUMMARY

An advantage of some aspects of the invention is to provide a biological information measurement apparatus capable of improving biological information detection precision, and the invention can be implemented in the following aspects or application examples:

### Application Example 1

A biological information measurement apparatus according to this application example includes a case section including a biological information detector that detects biological information on a user, a band section that attaches the case section to the user, a first electrode provided on a contact surface of the case section or the band section that comes into contact with the user's body, a pinched section provided as part of the case section or the band section and pinched by the user with fingers, a second electrode provided on the pinched section, and an electrocardiogram detector that uses the first electrode and the second electrode to detect the user's electrocardiogram.

According to this application example, in which the electrocardiogram detector that uses the first electrode and the second electrode to detect the user's electrocardiogram is provided, the first electrode keeps in contact with the user's body (skin) is maintained in a state in which the band section attaches the case section to the user's body, and the second electrode provided on the pinched section comes into contact with the user's fingers (skin) when the user pinches the pinched section. In the state in which the user pinches the pinched section on which the second electrode is provided with fingers, the contact between the second electrode and the user's skin is more likely to be maintained in a stable manner than, for example, in a case where the second electrode is provided on part of the case section or the band section and the user touches the second electrode with a finger or a palm. The configuration suppresses the amount of error due to variation in electric potential possibly resulting from disturbing factors different from the user's electrocardiogram due to unstable contact between the second electrode and the user's skin, whereby a biological information measurement apparatus capable of acquiring an accurate electrocardiogram can be provided.

### Application Example 2

In the biological information measurement apparatus according to the application example described above, it is preferable that the second electrode is provided on each principal contact surface of the pinched section that comes into contact with the skin.

According to this present application example, in which the second electrode is provided on each surface of the pinched section pinched with the user's fingers, the contact between the second electrode and the user's skin is satisfactorily maintained, whereby an accurate electrocardiogram with the amount of error suppressed can be produced.

### Application Example 3

In the biological information measurement apparatus according to the application example described above, it is preferable that the band section has one end connected to the case section and another end that is a free end, and that the pinched section is arranged at the other end of the band section.

According to this application example, which has the configuration in which the other end of the band section is formed as the pinched section and the second electrode is provided on the pinched section, the pinched section (second electrode) that allows acquisition of a stable electrocardiogram can be provided with no increase in the number of parts.

### Application Example 4

In the biological information measurement apparatus according to the application example described above, it is preferable that the pinched section has a connected end connected to the case section or the band section and a free end capable of releasing connection thereof to the case section or the band section.

According to this application example, to acquire an electrocardiogram by using the pinched section on which the second electrode is provided, the free end of the pinched section connected to the case section or the band section is released to allow the user to pinch the pinched section, whereas when no electrocardiogram is acquired, the free end of the pinched section can be connected to the case section or the band section. Therefore, when no electrocardiogram is acquired, the pinched section is advantageously unlikely to bother the user.

### Application Example 5

It is preferable that the biological information measurement apparatus according to the application example described above further includes a pinched section accommodating section having an accommodating mechanism capable of adjusting a length of the pinched section.

According to this application example, when the second electrode is not used (no electrocardiogram is acquired), the accommodating mechanism is used to accommodate at least part of the pinched section in the pinched section accommodating section, whereas when the second electrode is used to acquire an electrocardiogram, the accommodating mechanism allows the user to pull out the pinched section accommodated in the pinched section accommodating section by a length that allows the user to pinch the pinched section. Therefore, during a period other than acquisition of an electrocardiogram, the pinched section is advantageously unlikely to bother the user.

Further, the configuration in which the accommodating mechanism allows the portion of the pinched section where the second electrode is formed to be accommodated in the pinched section accommodating section can reduce the amount of dirt that adheres to the second electrode when it is not in use, whereby an increase in contact resistance of the second electrode that could contribute to an error in an electrocardiogram can be suppressed for continuously acquisition of accurate electrocardiograms containing a small amount of error.

### Application Example 6

In the biological information measurement apparatus according to the application example described above, it is preferable that the accommodating mechanism is capable of adjusting the length of the pinched section in such away that the length of the pinched section in a case where the second electrode is used is greater than the length of the pinched section in a case where the second electrode is not used.

According to this application example, the length of the pinched section can be so adjusted that when the second electrode is used (when electrocardiogram is acquired), the state in which the pinched section is allowed to be pinched with fingers is achieved, whereas when the second electrode is not used, the state in which the pinched section does not bother the user is achieved.

### Application Example 7

In the biological information measurement apparatus according to the application example described above, it is preferable that the accommodating mechanism is formed of a reel mechanism or a slide mechanism.

According to this application example, the accommodating mechanism can be configured to be capable of adjusting the length of the pinched section in such away that when the second electrode is not used, the pinched section is accommodated in the pinched section accommodating section, whereas when the second electrode is used, the pinched section can be pulled out from the pinched section accommodating section by a length that allows the user to pinch the pinched portion so that the portion where the second electrode is formed can be pulled out from the pinched section accommodating section and pinched.

### Application Example 8

In the biological information measurement apparatus according to the application example described above, it is preferable that the pinched section is provided as part of the band section along a direction in which the band section extends and a direction in which the pinched section extends.

According to this application example, the pinched section on which the second electrode is provided is provided as part of the band section along the direction in which bands of the band section extend and the direction in which the pinched section extends. As a result, since the longitudinal direction of the pinched section coincides with the direction in which the band section extends, the pinched section can be provided in a space efficient manner, whereby the pinched section is advantageously unlikely to bother the user when the second electrode (pinched section) is not used.

### Application Example 9

In the biological information measurement apparatus according to the application example described above, it is preferable that a third electrode insulated from the first electrode is provided on the surface on which the first electrode is arranged.

According to this application example, in the state in which the band section attaches the case section to the user's body, the third electrode, which keeps in contact with the user's skin, is so arranged along with the first electrode as to be insulated from the first electrode. For example, an electrocardiograph using the first and second electrode and satisfactorily grounded can be formed by arranging a ground electrode (ground) as the third electrode, whereby a more accurate electrocardiogram can be obtained.

### Application Example 10

In the biological information measurement apparatus according to the application example described above, it is preferable that the third electrode is an indifferent electrode.

According to this application example, in the state in which the band section attaches the case section to the user's body, the indifferent electrode, which keeps in contact with the user's skin, is so arranged along with the first electrode as to be insulated from the first electrode. In this state, when the user pinches the second electrode provided on the pinched section so that the second electrode comes into contact with the user's skin, an electrocardiograph using the first and second electrode and using the indifferent electrode as a reference electric potential can be formed. As a result, an advantageous effect of suppression of variation in the base line of an electrocardiogram (fluctuation in waveform) and hence acquisition of a more accurate electrocardiogram is provided.

### Application Example 11

It is preferable that the biological information measurement apparatus according to the application example described above further includes a pulse wave detector that is provided on the contact surface of the case section that comes into contact with the user's body and detects the user's pulse wave.

According to this application example, the user's pulse wave can be detected as well as the user's electrocardiogram. The number of types of detected biological information on the user therefore increases, whereby the versatility and convenience of the biological information measurement apparatus can be improved.

### Application Example 12

In the biological information measurement apparatus according to the application example described above, it is preferable that, in a cross-sectional view viewed along a direction parallel to the contact surface of the case section that comes into contact with the user's body, the pulse wave detector is provided on a protrusion that protrudes from the case section toward the user's skin, and that at least part of the first electrode is provided on the protrusion.

According to this application example, in the state in which the biological information measurement apparatus is attached to the user, the pulse wave detector and the first electrode, which are provided on the protrusion that protrudes toward the user's skin, are in contact with the user's skin in a stable manner. A more accurate electrocardiogram and pulse wave can therefore be advantageously acquired.

### Application Example 13

It is preferable that the biological information measurement apparatus according to the application example described above further includes a detection window which is provided through the surface on which the first electrode is arranged and through which the user's pulse wave is detected.

According to this application example, since the detection window, which is provided through the surface on which the first electrode is arranged, allows detection of the user's pulse wave as well as the user's electrocardiogram, the number of types of detected biological information on the user increases, whereby a biological information measurement apparatus having high versatility and convenience can be provided.

### Application Example 14

In the biological information measurement apparatus according to the application example described above, it is preferable that, in a side view viewed along a direction parallel to the contact surface of the case section that comes into contact with the user's body, the case section has a protrusion that is adjacent to the detection window and protrudes from the case section toward the user's skin, and at least part of the first electrode is provided on the protrusion.

According to this application example, the pulse wave detector and the first electrode, which are provided on the protrusion that protrudes toward the user's skin, are in contact with the user's skin in a stable manner, whereby a more accurate electrocardiogram and pulse wave can be obtained.

### Application Example 15

In the biological information measurement apparatus according to the application example described above, the second electrode preferably has a recess.

According to this application example, when the user pinches the pinched section with fingers, the recess of the second electrode follows the fingers, and the contact between the second electrode and the fingers is therefore satisfactorily maintained, whereby a more accurate electrocardiogram can be obtained.

### BRIEF DESCRIPTION OF THE DRAWINGS

The invention will be described with reference to the accompanying drawings, wherein like numbers reference like elements.

FIG. 1A is a front view showing a biological information measurement apparatus according to a first embodiment of the invention, and FIG. 1B is a partial cross-sectional view taken along the line a-a in FIG. 1A.

FIG. 2 is a rear view showing the biological information measurement apparatus in the first embodiment.

FIG. 3 is a block diagram showing the configuration of the biological information measurement apparatus in the first embodiment.

FIG. 4 is a block diagram showing the configuration of a measurement section in the first embodiment.

FIG. 5 is a block diagram showing the configuration of an electrocardiogram measurement section in the first embodiment.

FIG. 6 is a block diagram showing the configuration of a controller in the first embodiment.

FIG. 7 shows an example of the waveforms of a pulse wave and an electrocardiogram in the first embodiment.

FIG. 8A shows an example of an electrocardiographic waveform in a biological information measurement apparatus of related art, and FIG. 8B shows an example of an electrocardiographic waveform in the first embodiment.

FIG. 9 is a diagrammatic view showing a state in which the biological information measurement apparatus in the first embodiment is attached (biological information measurement state).

FIGS. 10A and 10B show a biological information measurement apparatus according to a second embodiment of the invention with the measurement apparatus attached. FIG. 10A is a diagrammatic view showing a non-measurement state, and FIG. 10B is a diagrammatic view showing a measurement state.

FIGS. 11A and 11B show a biological information measurement apparatus according to a third embodiment of the invention with the measurement apparatus attached. FIG. 11A is a diagrammatic view showing a non-measurement state, and FIG. 11B is a diagrammatic view showing a measurement state.

FIG. 12 is a diagrammatic view showing a biological information measurement apparatus according to a fourth embodiment of the invention with the measurement apparatus attached (with measurement apparatus ready for measurement).

FIGS. 13A and 13B show Variation 1 of the biological information measurement apparatus. FIG. 13A is a diagrammatic view showing a non-measurement state, and FIG. 13B is a diagrammatic view showing a measurement state.

FIGS. 14A and 14B show Variation 2 of the biological information measurement apparatus. FIG. 14A is a diagrammatic view showing a non-measurement state, and FIG. 14B is a diagrammatic view showing a measurement state.

FIG. 15 is a rear view showing a biological information measurement apparatus according to a fifth embodiment of the invention.

FIG. 16 is a cross-sectional view showing a main body section and a light transmissive member in the fifth embodiment.

FIG. 17 is a rear view of a biological information measurement apparatus showing another aspect of the arrangement and shape of each electrode in the fifth embodiment.

FIG. 18 is a cross-sectional view taken along the line B-B in FIG. 17.

### DESCRIPTION OF EXEMPLARY EMBODIMENTS

Embodiments of the invention will be described below with reference to the drawings. In the following drawings, each layer and each member are so drawn at scales different from actual scales in some cases as to be large enough to be recognizable.

### First Embodiment

### Overall Configuration of Biological Information Measurement Apparatus

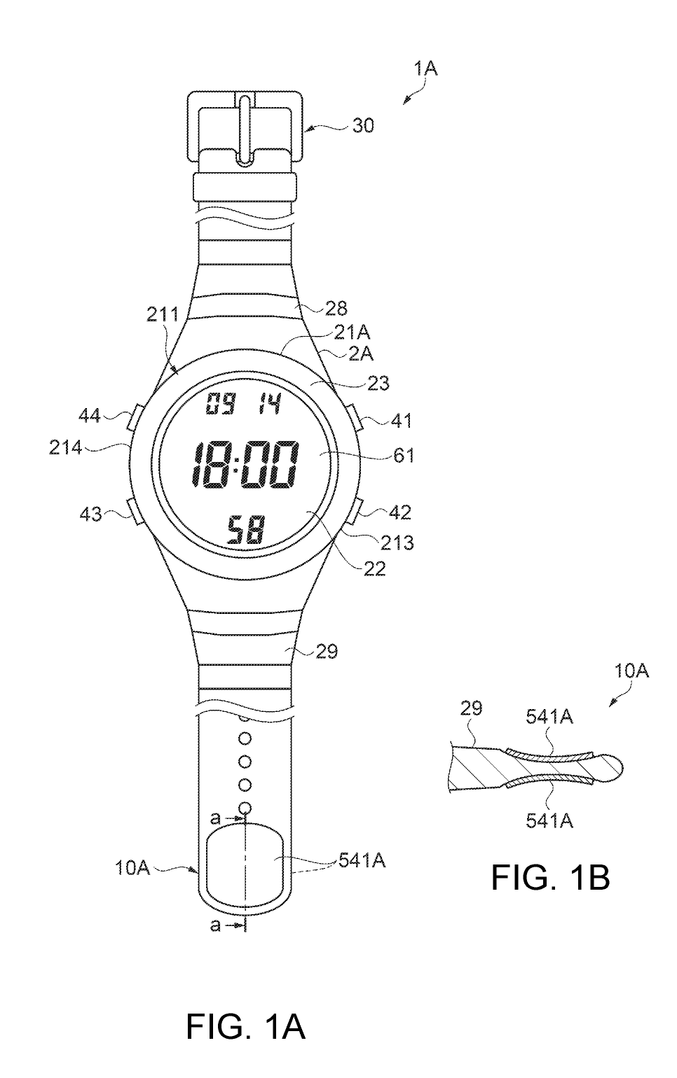

An overall configuration of a biological information measurement apparatus will first be described. FIG. 1A is a front view showing a biological information measurement apparatus according to a first embodiment of the invention, and FIG. 1B is a partial cross-sectional view taken along the line a-a in FIG. 1A.

In FIGS. 1A and 1B, a biological information measurement apparatus (hereinafter abbreviated to measurement apparatus in some cases) 1A according to the present embodiment is a wristwatch-shaped wearable apparatus attached to a user's wrist or any other attachment site for use and detects and stores biological information on the user. Specifically, the measurement apparatus 1A detects a pulse wave and an electrocardiogram as the biological information on the user, and not only stores the electrocardiogram but also calculates the pulse rate on the basis of the detected pulse wave and stores the calculated pulse rate.

The thus configured measurement apparatus 1A includes a case section 2A having a main body section 21A, a pair of bands 28 and 29, and an apparatus main body 3 accommodated in the case section 2A.

The pair of bands 28 and 29 correspond to a band section that is an attachment member according to the embodiment of the invention, are connected to one end and the other end of the case section 2A (main body section 21A) respectively in the direction in which the case section 2A extends (longitudinal direction), and extend in opposite directions from the case section 2A. The pair of bands 28 and 29 are so configured as to be fixable to each other via a buckle 30, which is provided at the front end of the band 28 (the end on the opposite side of the case section 2A with respect to the portion where the band 28 is connected to the case section 2A). Fixing the bands 28 and 29 to each other as described above allows the case section 2A to be attached to the attachment site described above. The bands 28 and 29 may instead be integrated with the case section 2A.

The band 28 (first band) has a front end portion (the end opposite one end connected to the case section 2A or the other end) provided with a pinched section 10A having a shape readily pinched by the user with fingers. As the shape readily pinched by the user with fingers, in the present embodiment, each surface (principal surface) of the pinched section 10A is so formed as to be concavely curved so that the surface readily follows the shape of a finger. In other words, part of the pinched section 10A is depressed, or the pinched section 10A has a recess. On the surfaces of the pinched section 10A that are pinched with fingers are arranged second electrodes 541A of a plurality of electrodes that form an electrocardiogram measurement section 54 of a measurement section 5, which will be described later. In the present embodiment, the second electrodes 541A are provided on opposite surfaces of the pinched section 10A that come into contact with the user's skin. Pinching an object with fingers refers to bringing a plurality of fingers into contact with different surfaces of the object or different positions thereon and means action of pinching the object with the plurality of fingers or achieving a state in which the object is not separate from the fingers.

The main body section 21A of the case section 2A accommodates the apparatus main body 3, which will be described later. The main body section 21A has a rear surface 212 (first surface), which is a surface that comes into contact with the user's body when the measurement apparatus 1A is attached to the user's body, a front surface 211 (second surface), which is a surface facing away from the rear surface 212, and a right side surface 213 and a left side surface 214, which connect the rear surface 212 and the front surface 213 to each other. That is, the rear surface 212 of the main body section 21A is a surface that comes into contact with the user's body (skin), a surface on which a first electrode 5421 and a pulse wave sensor 531 of a pulse wave detector 53, which will be described later, are arranged, or a surface on which a light transmissive member that covers the pulse wave sensor 531 is arranged.

On the other hand, the front surface 211 is a surface facing away from the rear surface 212, a surface having a display section 61, which will be described later, or a rear surface with reference to the user's skin touching surface of the case section 2A or the main body section 21A. That is, the front surface 211 is a surface most remote from the user's skin.

In the measurement apparatus having the surfaces described above, the display section 61, which forms the apparatus main body 3, is provided in a roughly central portion of the front surface 211, and the display section 61 is covered with a circular cover 22. The front surface 211 is one of the surfaces opposite the rear surface 212 when viewed along a normal to a display surface of the display section 61. The front surface 211 may be a single flat surface or may partially have a curved surface or a recess and a protrusion.

The front surface 211 is provided with an annular bezel 23, which surrounds the display section 61 and the cover 22.

Buttons 41 to 44 of an operation section 4, which forms the apparatus main body 3, are arranged on the right side surface 213 and the left side surface 214. Each of the buttons 41 to 44 is a button that comes out and retracts relative to the main body section 21A.

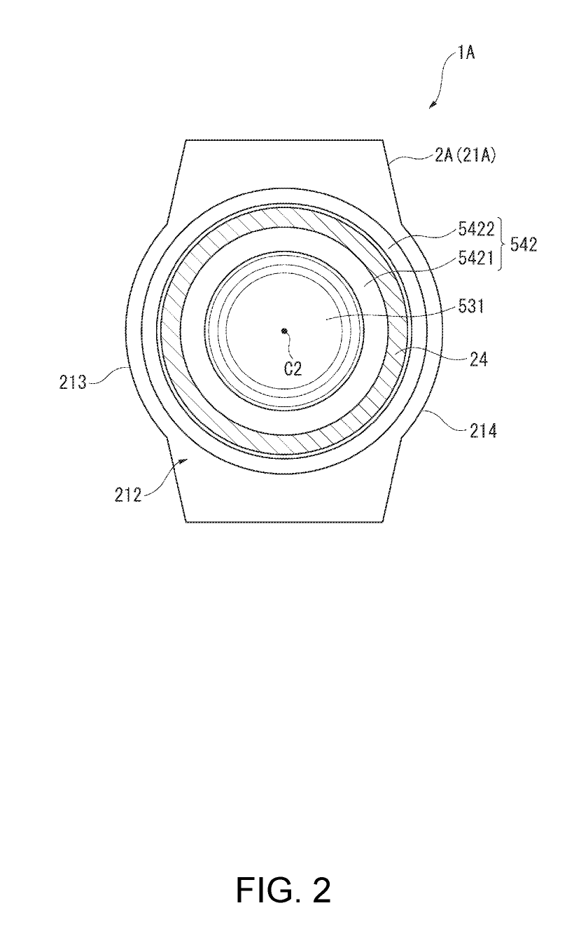

FIG. 2 is a rear view showing the measurement apparatus 1A and specifically shows the rear surface 212 of the main body section 21A.

The rear surface 212 (corresponding to second surface) is a surface facing the attachment site described above when the measurement apparatus 1A is attached to the attachment site (surface that comes into contact with user's skin in attached state). On the rear surface 212 are arranged a rear-surface-side electrode 542, which forms the electrocardiogram measurement section 54, and the pulse wave sensor 531.

The rear-surface-side electrode 542 has two electrodes, the first electrode 5421 and an indifferent electrode 5422 in the present embodiment. The first electrode 5421 has a roughly circular shape and is so disposed as to be exposed and surrounds the pulse wave sensor 531. The indifferent electrode 5422 has a roughly circular shape and is so disposed as to be exposed and surrounds the first electrode 5421 via an insulator 24. The indifferent electrode 5422 is therefore so arranged as to be insulated from the first electrode 5421. The first electrode 5421 and the indifferent electrode 5422 are concentrically disposed around the center C2 of the circular pulse wave sensor 531.

The pulse wave sensor 531 is a roughly circular sensor that forms the pulse wave detector 53 of the measurement section 5, and the pulse wave sensor 531 is disposed roughly at the center of the rear surface 212. Instead, the pulse wave sensor 531 may be directly arranged on the rear surface 212 or may be provided in the apparatus main body 3 provided in the main body section 21A, and the light transmissive member, which covers a light emitting device and a light receiving device of the pulse wave sensor 531, may be attached to the rear surface 212.

### Configuration of Apparatus Main Body

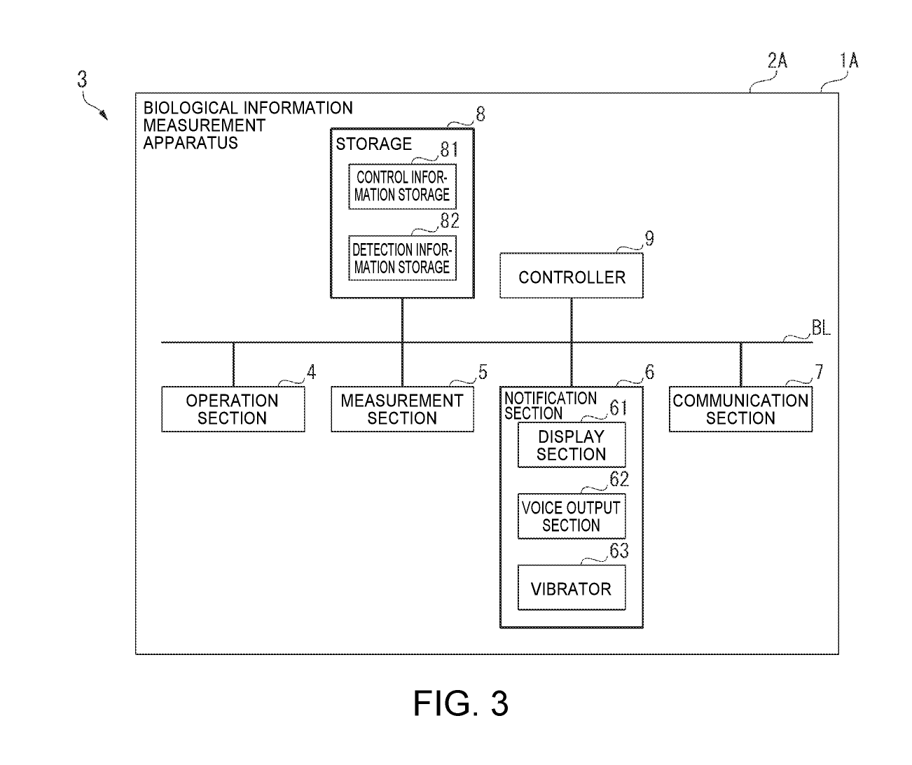

FIG. 3 is a block diagram showing the configuration of the measurement apparatus 1A.

The apparatus main body 3 includes the operation section 4, the measurement section 5, a notification section 6, a communication section 7, a storage 8, and a controller 9, as shown in FIG. 3.

### Configuration of Operation Section

The operation section 4 has the buttons 41 to 44 described above and outputs operation signals according to input operations performed on the buttons 41 to 44 to the controller 9. The operation section 4 does not necessarily have the configuration including buttons and may have a configuration including a touch panel arranged on the display section 61 of the notification section 6, which will be described later, or a configuration in which the user's tapping operation is detected.

### Configuration of Measurement Section

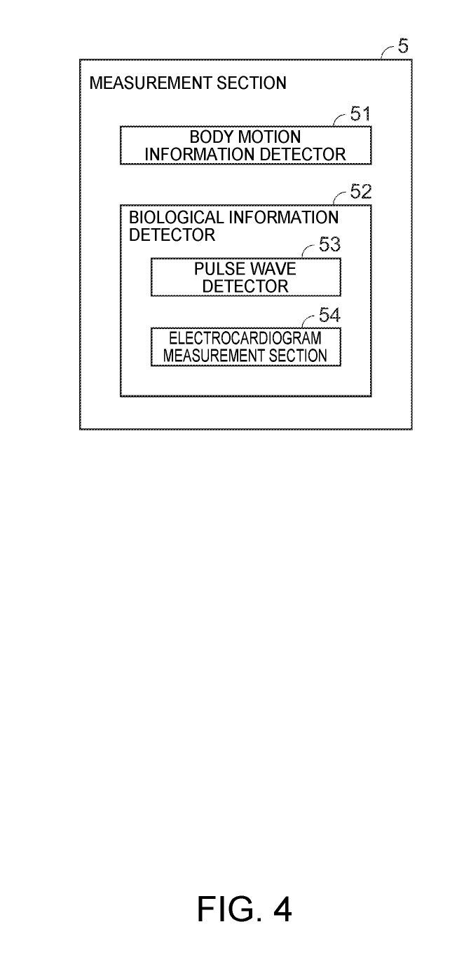

FIG. 4 is a block diagram showing the configuration of the measurement section 5.

The measurement section 5 includes a body motion information detector 51 and a biological information detector 52, each of which operates under the control of the controller 9.

The body motion information detector 51 detects body motion information representing the user's body motion and outputs the body motion information to the controller 9. In the present embodiment, the body motion information detector 51 includes a body motion sensor, for example, an acceleration sensor and detects, as the body motion information, an acceleration signal that changes in accordance with the user's body motion. The body motion information detector 51 may further detect angular velocity that changes in accordance with the user's body motion as well as the acceleration.

### Configuration of Biological Information Detector

The biological information detector 52 detects the user's biological information. In the present embodiment, the biological information detector 52 includes the electrocardiogram measurement section 54 and the pulse wave detector 53.

### Configuration of Pulse Wave Detector

The pulse wave detector 53 includes the pulse wave sensor 531 described above and detects the user's pulse wave under the control of the controller 9. The pulse wave sensor 531 is a photoelectric sensor including, although not shown, a light emitting device, such as an LED (light emitting diode), a light receiving device, such as a photodiode, and the light transmissive member, which covers the light emitting device and the light receiving device. In the pulse wave sensor, light emitted from the light emitting device toward the user's body travels via a blood vessel in the user's body and is received with the light receiving device. A signal representing a temporal change in the amount of light received with the light receiving device is outputted as a pulse wave signal to the controller 9, which will be described later, and the controller 9 analyzes the pulse wave signal to calculate the pulse rate.

### Configuration of Electrocardiogram Measurement Section

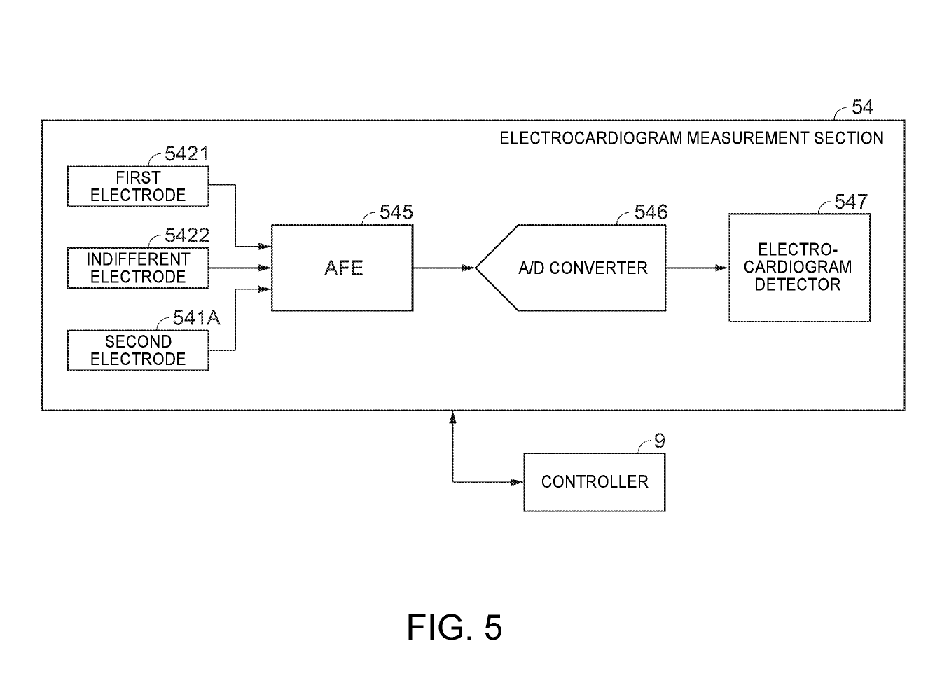

FIG. 5 is a block diagram showing the configuration of the electrocardiogram measurement section 54.

The electrocardiogram measurement section 54 detects the user's electrocardiogram and outputs an electrocardiographic signal representing the electrocardiogram to the controller 9. The electrocardiogram measurement section 54 includes an AFE (analog front end) 545, an A/D converter 546, and an electrocardiogram detector 547 as well as the first electrode 5421 and the indifferent electrode 5422 (rear-surface-side electrode 542) and the second electrode 541A described above, as shown in FIG. 5. The controller 9, which controls the measurement apparatus 1A, is connected to the electrocardiogram measurement section 54.

In the electrocardiogram measurement section 54, the electrocardiogram detector 547 uses, under the control of the controller 9, the second electrodes 541A, the first electrode 5421, and the indifferent electrode 5422 to detect an electrocardiogram of the user to which the measurement apparatus 1A is attached. Specifically, an unstable, minute-level analog signal detected through the second electrodes 541A, the first electrode 5421, and the indifferent electrode 5422 is inputted to the AFE 545, where the analog signal undergoes frequency band limitation and signal amplification, and the resultant signal is outputted to the A/D converter 546. The A/D converter 546 A/D-converts the analog signal inputted from the AFE 545 and outputs the resultant digital signal to the electrocardiogram detector 547.

The electrocardiogram detector 547 performs signal processing in which it processes the signal inputted from the A/D converter 546 and outputs an electrocardiographic signal based on the processed signal to the controller 9. Specifically, the electrocardiogram detector 547 filters the inputted signal for noise removal and outputs the resultant electrocardiographic signal to the controller 9.

### Configuration of Notification Section

Referring back to FIG. 3, the notification section 6 notifies the user of a variety of types of information under the control of the controller 9. The notification section 6 includes the display section 61, a voice output section 62, and a vibrator 63.

The display section 61 has any of a variety of display panels, such as a liquid crystal panel, and displays information inputted from the controller 9. For example, the display section 61 displays body motion information and biological information (electrocardiogram and pulse rate) detected and analyzed by the measurement section 5 described above. The display section 61 further displays presentation information generated by the controller 9.

The voice output section 62 includes a voice output device, such as a loudspeaker, and outputs voice according to a voice signal inputted from the controller 9.

The vibrator 63 has a motor that operates under the control of the controller 9 and notifies the user of, for example, an alarm in the form of vibration generated by the driven motor.

The notification section 6 only needs to include at least one of the display section 61, the voice output section 62, and the vibrator 63 described above.

### Configuration of Communication Section

The communication section 7 includes a communication module capable of communicating with an external apparatus. The communication section 7 transmits detected and measured body motion information and biological information to the external apparatus on a regular basis and outputs information received from the external apparatus to the controller 9. In the present embodiment, the communication section 7 wirelessly communicates with the external apparatus on the basis of a short-range wireless communication scheme. The communication section 7 may instead communicate with the external apparatus via a relay apparatus, such as a cradle, or a cable. The communication section 7 may still instead communicate with the external apparatus over a network.

### Configuration of Storage

The storage 8 is formed of a storage device, such as a flash memory, and includes a control information storage 81 and a detection information storage 82.

The control information storage 81 stores a variety of programs, data, and other types of control information necessary for the action of the measurement apparatus 1A. As examples of the programs, a control program that controls the measurement apparatus 1A and an electrocardiogram measurement program that allows execution of electrocardiogram measurement are stored.

The detection information storage 82 stores the body motion information and biological information detected by the measurement section 5 described above and results of analysis of the body motion information and biological information (pulse rate and electrocardiogram, for example) performed by the controller 9. The detection information storage 82 is configured to sequentially store the information described above and overwrite the information stored at the earliest point of time with newly acquired information in a case where the detection information storage 82 is short of storage capacity.

### Configuration of Controller

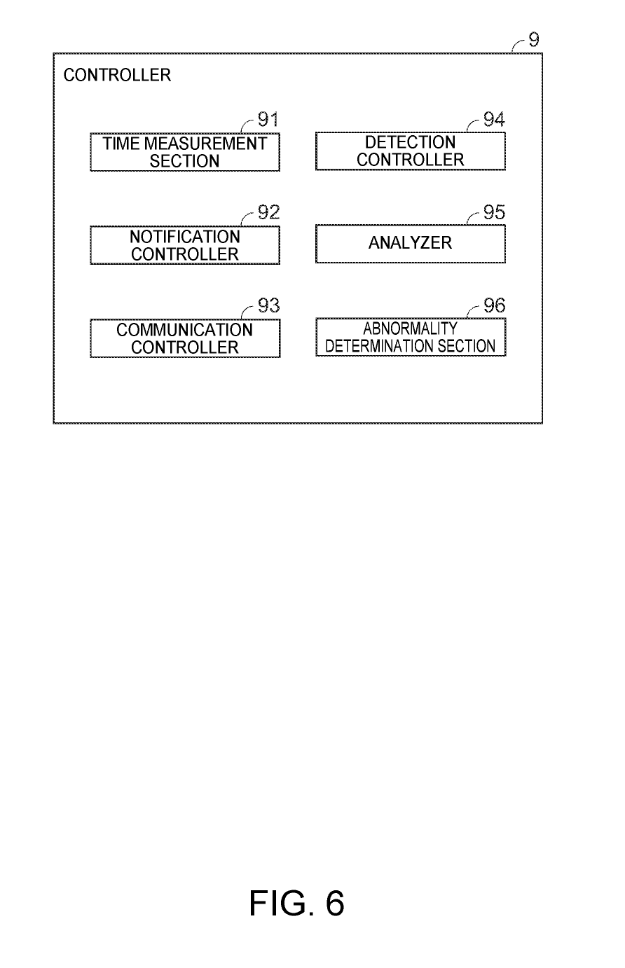

FIG. 6 is a block diagram showing the configuration of the controller 9.

The controller 9 has a processing circuit and controls the action of the measurement apparatus 1A autonomously or in accordance with an operation signal inputted from the operation section 4 described above. The controller 9, for example, controls the measurement section 5 described above to cause it to detect body motion information and biological information. In this process, when causing the electrocardiogram measurement section 54 to detect and measure the user's electrocardiogram, the controller 9 causes the electrocardiogram measurement section 54 to perform temporary electrocardiogram measurement.

The thus configured controller 9 has, as functional sections achieved by causing the processing circuit described above to execute a program stored in the control information storage 81, a time measurement section 91, a notification controller 92, a communication controller 93, a detection controller 94, an analyzer 95, and an abnormality determination section 96, as shown in FIG. 6.

### Configurations of Time Measurement Section, Notification Controller, and Communication Controller

The time measurement section 91 measures the current date and time.

The notification controller 92 controls the action of the notification section 6. For example, the notification controller 92 causes the notification section 6 to notify the state of the action of the measurement apparatus 1A and presentation information including display and voice representing, for example, a result of the detection performed by the measurement section 5. The notification controller 92 further causes the vibrator 63 to drive the motor thereof as required to cause the vibrator 63 to notify predetermined information in the form of vibration generated by the driven motor.

The communication controller 93 controls the action of the communication section 7 described above.

### Configuration of Detection Controller

The detection controller 94 controls the action of the measurement section 5 described above. For example, the detection controller 94 causes the body motion information detector 51 to detect the user's body motion and further causes the pulse wave detector 53 to detect the user's pulse wave. The detection controller 94 then causes the detection information storage 82 described above to store an acceleration signal representing the body motion and a pulse wave signal representing the pulse wave along with the current date and time described above.

The detection controller 94 further causes the electrocardiogram measurement section 54 to perform temporary electrocardiogram measurement. The detection controller 94 then causes the electrocardiogram measurement section 54 to perform electrocardiogram measurement (final measurement) and causes the detection information storage 82 to store an electrocardiographic signal representing the measured electrocardiogram along with the current date and time described above. The detection controller 94 may instead cause the detection information storage 82 to store, as the biological information, the pulse rate calculated on the basis of the pulse wave signal along with the current date and time.

### Configuration of Analyzer

The analyzer 95 analyzes the body motion information and biological information inputted from the body motion information detector 51 and the biological information detector 52, respectively.

Specifically, the analyzer 95 calculates the user's pulse rate on the basis of the pulse wave signal inputted from the pulse wave detector 53 and the acceleration signal inputted from the body motion information detector 51. For example, the analyzer 95 removes body motion noise components based on the acceleration signal from the pulse wave signal to generate a beat signal. The analyzer 95 then performs frequency analysis, such as FFT (Fast Fourier Transform), on the beat signal, extracts the frequency of the pulse from a result obtained from the analysis (power spectrum), and calculates the pulse rate on the basis of the pulse frequency. The analyzer 95 does not necessarily calculate the pulse rate as described above and may instead calculate the pulse rate on the basis of another approach.

The analyzer 95 generates an RR waveform signal representing a temporal change in RR interval (time difference between sharpest-peak R wave contained in pulse wave signal and preceding R wave) for each frame on the basis of a result of the frequency analysis described above. The analyzer 95 further calculates a heartrate variation coefficient CVRR, which is an RR interval variation coefficient, to generate a variation coefficient waveform signal representing a temporal change in the heartrate variation coefficient CVRR.

The analyzer 95 further calculates the user's walking pace (pitch) on the basis of the acceleration signal. For example, the analyzer 95 performs the same frequency analysis described above on the acceleration signal, extracts the frequency of body motion from a result obtained from the analysis, and calculates the walking pace on the basis of the body motion frequency.

The analyzer 95 additionally analyzes the electrocardiographic signal inputted from the electrocardiogram measurement section 54.

The analyzer 95 then causes the detection information storage 82 described above to store the calculated pulse rate and walking pace and the electrocardiogram analysis result.

### Configuration of Abnormality Determination Section

The abnormality determination section 96 determines whether or not an abnormality classified into arrhythmia has occurred in the user on the basis of the RR waveform signal and the variation coefficient waveform signal generated by the analyzer 95 and the pulse rate calculated by the analyzer 95. Examples of the arrhythmia may include atrial fibrillation, extrasystole, tachycardia, and bradycardia.

Atrial fibrillation refers to a state in which the beat rate of an atrium increases to 300 or greater per minute and the heart beats irregularly and quickly, resulting in stagnation of blood in the heart. When atrial fibrillation occurs, the amplitude of the RR waveform signal described above increases, and the heartrate variation coefficient CVRR described above greatly changes. Accordingly, the abnormality determination section 96 determines whether or not atrial fibrillation has occurred on the basis of these phenomena. However, the abnormality determination section 96 does not necessarily make the determination as described above and may instead use another method to determine whether or not atrial fibrillation has occurred. For example, the abnormality determination section 96 may perform matching of the waveform of the pulse wave signal described above with the waveform of a pulse wave signal obtained when atrial fibrillation occurred in the past, and the abnormality determination section 96 may determine that atrial fibrillation has occurred when a result of the matching shows that the two waveforms are roughly the same.

Extrasystole refers to a state in which an abnormal stimulus causes the heart to beat in a cycle different from a normal cycle, resulting in premature systole. When extrasystole occurs, the pulse wave signal contains a waveform different from the waveform of a normal sinus rhythm. The abnormality determination section 96 therefore performs matching of the waveform obtained when extrasystole occurs with the waveform of an acquired pulse wave signal. When a result of the matching shows that the two waveforms are roughly the same, the abnormality determination section 96 determines that extrasystole has occurred. The waveform obtained when extrasystole occurs may be an average waveform or the waveform of extrasystole that occurred in the past in the user.

Tachycardia refers to a state of an abnormally fast pulse, and bradycardia refers to a state of an abnormally slow pulse. For example, tachycardia is suspected to have occurred when the pulse rate of a typical adult, which ranges from 60 to 70 bpm at rest, is higher than 100 bpm except the time of exercise, and bradycardia is suspected to have occurred when the pulse of a typical adult is lower than or equal to 50 bpm.

When tachycardia occurs, a state in which the RR interval described above is shorter than a normal value continues, whereas when bradycardia occurs, a state in which the RR interval described above is longer than the normal value continues. The abnormality determination section 96 therefore determines that tachycardia has occurred when a state in which the RR interval is longer than a tachycardia threshold set in accordance with the user continues for a predetermined period and determines that bradycardia has occurred when a state in which the RR interval is shorter than a bradycardia threshold (threshold smaller than tachycardia threshold) set in accordance with the user continues for a predetermined period.

When the thus configured abnormality determination section 96 determines that an abnormality classified into arrhythmia has occurred in the user, the notification controller 92 described above causes the notification section 6 to notify presentation information that prompts the user to measure an electrocardiogram. For example, the notification controller 92 causes the display section 61 to display a message that prompts the user to measure an electrocardiogram. In addition, for example, the notification controller 92 causes the voice output section 62 to output predetermined voice (alarm sound, for example) or cause the vibrator 63 to generate the vibration described above.

### Electrocardiogram Measurement

Biological information measurement performed by the measurement apparatus 1A according to the present embodiment will next be described. In particular, advantageous effects provided by electrocardiogram measurement will be described in detail.

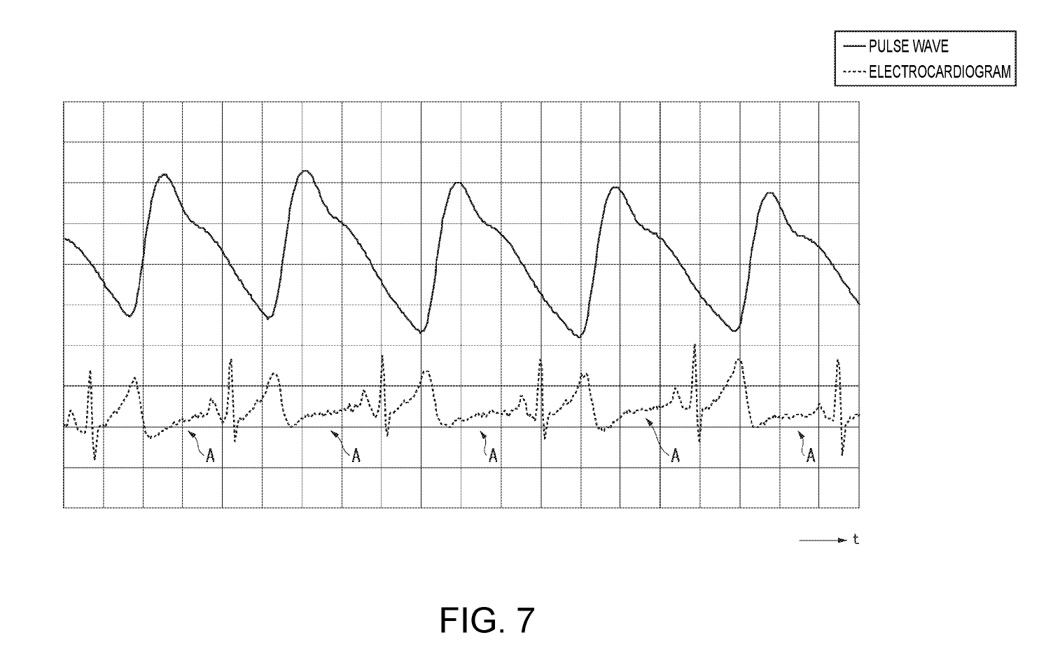

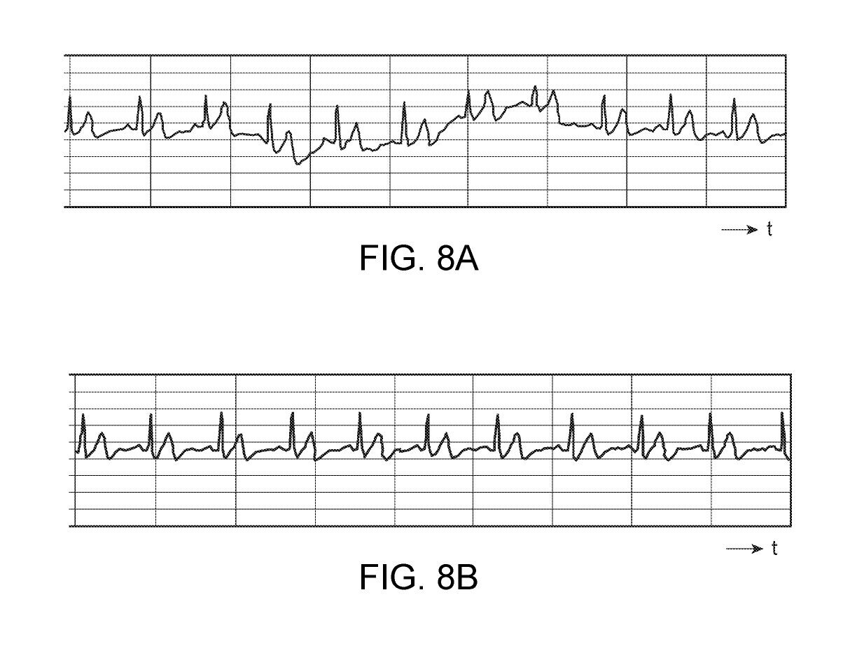

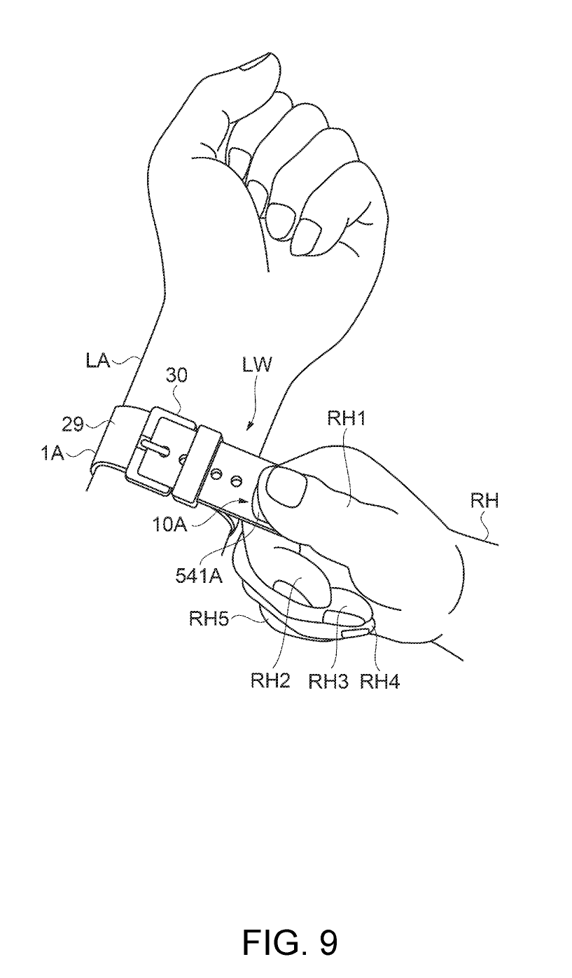

FIG. 7 shows an example of the waveforms of a measured pulse wave and electrocardiogram. FIG. 8A shows an example of an electrocardiographic waveform measured by a biological information measurement apparatus of related art, and FIG. 8B shows an example of an electrocardiographic waveform measured by the biological information measurement apparatus 1A according to the present embodiment. FIG. 9 is a diagrammatic view showing a state in which the biological information measurement apparatus 1A according to the present embodiment is attached (biological information measurement state).

FIG. 7 shows an example of the waveforms of a pulse wave and an electrocardiogram measured by the biological information measurement apparatus 1A described above. The waveforms (portions indicated by arrows A) of the pulse wave and the electrocardiogram in FIG. 7 are normal waveforms containing no error or other types of noise. However, since electrocardiogram measurement, among a variety of types of biological information measurement, requires signal components over a particularly wide frequency band (0.1 to 40 Hz, for example), an unstable state in which the measurement electrodes (first electrode 5421 and second electrodes 541A) are in contact with the user's body may undesirably disturb the resultant electrocardiographic waveform due to influence of frequencies of disturbing factors that differ from the electrocardiographic frequency. The configuration of an electrocardiogram measurement section of related art that tends to cause the unstable contact state between the measurement electrodes and the user's body will be described and advantageous effects provided by the electrocardiogram measurement section 54 (see FIG. 5) of the present embodiment will be then described.

As the electrocardiogram measurement section of a measurement apparatus of related art, there is a known configuration in which the pair of measurement electrodes are provided as follows: The first electrode is provided on the case section or the band section, specifically, on the surface thereof that comes into contact with the user's body (skin); and the second electrode is provided on a surface different from the surface on which the first electrode is provided, for example, the side surface of the case section. In the measurement apparatus of related art, when it is attached to the user's arm and the first electrode keeps in contact with the user's body, touching the second electrode with a finger of the hand of the arm different from the arm to which the biological information measurement apparatus is attached allows electrocardiogram measurement. In the method for causing the user to press a finger against the second electrode, it is difficult to maintain the state in which the second electrode is in contact with the user's finger (skin) in a stable manner for a predetermined period (one minute, for example) required for the measurement, resulting in unstable contact between the second electrode and the user's body in many cases. The unstable contact state between the measurement electrodes and the user's body, for example, results in a large amount of disturbance of the base line of the electrocardiographic waveform due to variation in contact resistance, a decrease in S/N ratio due to electromagnetically induced noise and other types of noise from a commercial power supply, and other adverse effects. FIG. 8A shows an electrocardiographic waveform with a disturbed or fluctuating base line due to the unstable contact state described above between the measurement electrodes and the user's body.

### Advantageous Effects Provided by First Embodiment

In contrast, in the measurement apparatus 1A according to the present embodiment, the pinched section 10A, which has a shape readily pinched by the user with fingers, is formed at the front end portion of the band 28, and the second electrodes 541A are formed on opposite surfaces of the pinched section 10A that are pinched with the fingers (see FIGS. 1A and 1B). In a state in which the measurement apparatus 1A is attached to a wrist (left wrist) LW of the user's left arm LA, the first electrode 5421 and the indifferent electrode 5422, which form the rear-surface-side electrode 542 described above, keep in contact with the skin of the left wrist LW, as shown in FIG. 9. In this state, when the user pinches the pinched section 10A with the thumb RH1 and the index finger RH2 of the user's right hand RH, the fingers are allowed to be firmly in contact with the second electrodes 541A provided on opposite surfaces of the pinched section 10A. As a result, the contact state between the second electrodes 541A and the user's skin is likely to be maintained in a stable manner, as compared with the configuration of related art in which a finger presses the second electrode, resulting in suppression of disturbance of the electrocardiographic waveform due to electric potential variation resulting from disturbing factors different from the user's electrocardiogram, whereby a precise electrocardiogram can be obtained.

Further, in the measurement apparatus 1A according to the present embodiment, as the rear-surface-side electrode 542, which forms the electrocardiogram measurement section 54, the first electrode 5421, which is one of the measurement electrodes, and the indifferent electrode 5422 are arranged on the rear surface 212 of the case section 2A with the indifferent electrode 5422 insulated from the first electrode 5421. The indifferent electrode 5422 is an electrode arranged in a position remote from a site under electrocardiogram measurement in which the first electrode 5421 and the second electrodes 541A are used to induce an electrocardiographic signal, and the indifferent electrode 5422 is also an electrode so positioned as to receive no influence of electrical activity in the site under measurement. The indifferent electrode 5422, which receives no influence of electrical activity in the site under measurement, can be used as a reference electrode that serves as a reference of electric potential in electrocardiogram measurement. An electrocardiographic waveform can therefore be more accurately measured and recorded.

The measurement apparatus 1A according to the present embodiment described above, which includes the second electrodes 541A formed on the pinched section 10A and the indifferent electrode 5422 arranged on the same surface where the first electrode 5421 is arranged as the rear-surface-side electrode 542, advantageously allows measurement and recording of a precise electrocardiographic waveform with disturbance and fluctuation of the base line thereof suppressed, such as that shown in FIG. 8B.

Further, in the measurement apparatus 1A according to the present embodiment, a free end (the other end) of the band 28 is used as the pinched section 10A, and the second electrodes 541A are formed on the pinched section 10A. As a result, the pinched portion 10A (second electrodes 541A) that allows a stable electrocardiogram to be produced can be provided with no increase in the number of parts of the wristwatch-shaped measurement apparatus 1A.

In the present embodiment, the configuration in which the second electrodes 541A are provided on opposite surfaces of the pinched section 10A that come into contact with the skin is employed. Further, each of the surfaces of the pinched section 10A that come into contact with the skin (surfaces on which second electrodes 541A are formed) is concavely curved so that the surface readily follows the shape of a finger. The configurations described above allow the contact state between the second electrodes 541A and the user's skin to be maintained in a satisfactory manner, whereby a more accurate electrocardiographic waveform can be measured and recorded.

In the present embodiment, the second electrodes 541A are provided on opposite surfaces of the pinched section 10A that come into contact with the user's skin, but the second electrodes 541A are not necessarily provided this way. For example, a single second electrode may be provided on one of the surfaces of the pinched section 10A. In this case, the second electrode is preferably provided on the band 28, specifically, the surface thereof that does not come into contact with the user's skin. The configuration described above allows stable electrocardiogram measurement to be performed with a minimally required electrode, allowing reduction in apparatus cost.

### Second Embodiment

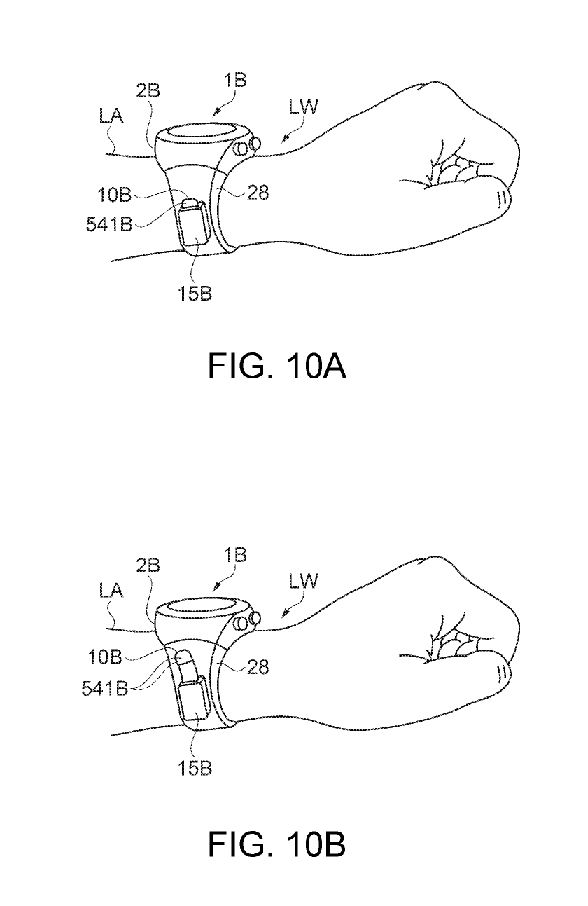

FIGS. 10A and 10B show a biological information measurement apparatus according to a second embodiment of the invention with the measurement apparatus attached. FIG. 10A is a diagrammatic view showing a non-measurement state, and FIG. 10B is a diagrammatic view showing a measurement state.

A biological information measurement apparatus 1B according to the present embodiment will be described with reference to FIGS. 10A and 10B. The same configuration portions as those in the first embodiment have the same numerals, and no duplicated description will be made.

The biological information measurement apparatus 1B according to the second embodiment shown in FIG. 10A includes a pinched section 10B and a pinched section accommodating section 15B, which has a pinched section accommodating mechanism that allows at least part of the pinched section 10B to be accommodated in the pinched section accommodating section 15B and the accommodated pinched section 10B to be pulled out by a predetermined amount from the pinched section accommodating section 15B, and the pinched section 10B and the pinched section accommodating section 15B are provided on the band 28, which is one of the bands 28 and 29, which attach a case section (main body section) 2B to a user. Pulling out the pinched section 10B means operation of increasing the amount of protrusion of the pinched section 10B from the pinched section accommodating section 15B and is operation of changing the length of the pinched section 10B exposed outside the pinched section accommodating section 15B.

The pinched section 10B has a shape readily pinched by the user with fingers, for example, a strip-like shape. The strip-shaped pinched section 10B has a connected end connected to the band 28 and a free end that is the end opposite the connected end, and second electrodes 541B are provided on the free end side. The second electrodes 541B are provided on opposite surfaces of the pinched section 10B (see FIG. 10B). The connected end of the pinched section 10B is connected to the pinched section accommodating mechanism, for example, a reel mechanism or a slide mechanism, and the pinched section accommodating mechanism allows at least part of the pinched section 10B on the connected end side to be accommodated in the pinched section accommodating section 15B (state shown in FIG. 10A) and part of the pinched section 10B on the free end side to be pulled out from the pinched section accommodating section 15B (state shown in FIG. 10B). The strip-shaped pinched section 10B is so arranged that the direction in which the strip-shaped pinched section 10B extends from the free end thereof to the connected end thereof (longitudinal direction) coincides with the direction in which the band 28 extends (longitudinal direction).

FIG. 10A shows the state in which no electrocardiogram is measured (second electrodes 541B are not used). Specifically, in a state in which the measurement apparatus 1B is attached to a wrist (left wrist) of the user's left arm LA, at least part of the pinched section 10B on the connected end side described above (major part of the pinched section 10B in the present embodiment) is accommodated in the pinched section accommodating section 15B by using the pinched section accommodating mechanism.

When the second electrodes 541B are used to measure an electrocardiogram, the pinched section 10B is pulled out by a predetermined amount from the pinched section accommodating section 15B by using the pinched section accommodating mechanism, and a state in which the second electrodes 541B on the pinched section 10B are allowed to be pinched with fingers of the user's right hand is achieved, as shown in FIG. 10B. The second electrodes 541B on the pinched section 10B are then pinched with the thumb RH1 and the index finger RH2 of the user's right hand RH (see FIG. 9), and an electrocardiogram is measured by using the first electrode (not shown), which forms the rear-surface-side electrode on the case section 2B, and the second electrodes 541B, as in the first embodiment described above.

As described above, the measurement apparatus 1B according to the present embodiment can provide the following advantageous effects as well as those provided by the first embodiment described above.

The measurement apparatus 1B according to the present embodiment has a structure in which when the second electrodes 541B are not used (when no electrocardiogram is measured), at least part of the pinched section 10B on the connected end side is accommodated in the pinched section accommodating section 15B by using the pinched section accommodating mechanism, whereas when the second electrodes 541B are used to measure an electrocardiogram, part of the pinched section 10B on the free end side accommodated in the pinched section accommodating section 15B is pulled out by a predetermined amount by using the pinched section accommodating mechanism. As a result, the pinched section 10B is advantageously unlikely to bother the user during a period other than measurement of an electrocardiogram.

Further, according to the configuration of the present embodiment in which the pinched section accommodating section 15B accommodates a major part of the pinched section 10B including the portion on which the second electrodes 541B are formed, the amount of dirt that adheres to and the magnitude of stress induced in the second electrodes 541B are reduced when the second electrodes 541B are not used, whereby an increase in contact resistance of the second electrodes 541B that could contribute to disturbance of a result (waveform) of electrocardiogram measurement is suppressed for continuous acquisition of accurate electrocardiograms containing a small amount of error.

Further, in the present embodiment, since the strip-shaped pinched section 10B is so arranged that the direction in which the pinched section 10B extends from the free end thereof toward the connected end thereof coincides with the direction in which the band 28 extends, the pinched section 10B can be provided in a space efficient manner, the pinched section 10B is advantageously more unlikely to bother the user when the second electrodes 541B (pinched section 10B) are not used (no electrocardiogram is measured).

Further, in the present embodiment, the configuration in which the pinched section 10B is pulled out in the same direction in which the front side of the case section 2B faces is employed. A configuration in which the pinched section 10B is pulled out in another direction may instead be employed. For example, a configuration in which the pinched section 10B is pulled out in the direction in which the band (first band) 28 extends from the case section 2B may be employed, or a configuration in which the pinched section 10B is pulled out in the direction perpendicular to the direction in which the band (first band) 28 extends from the case section 2B may be employed. Still instead, a configuration in which the pinched section 10B is pulled out in still another direction may be employed.

### Third Embodiment

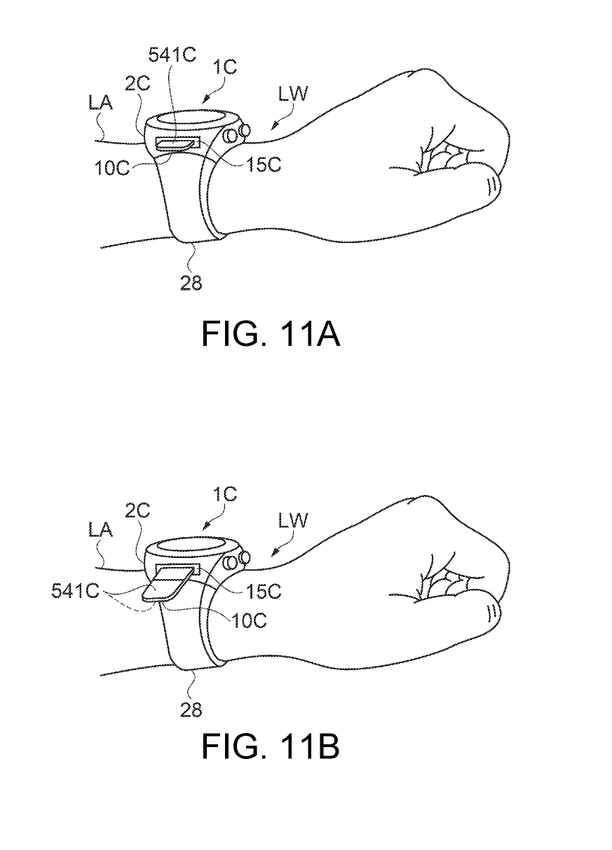

FIGS. 11A and 11B show a biological information measurement apparatus according to a third embodiment of the invention with the measurement apparatus attached. FIG. 11A is a diagrammatic view showing a non-measurement state, and FIG. 11B is a diagrammatic view showing a measurement state.

A biological information measurement apparatus 1C according to the present embodiment will be described with reference to FIGS. 11A and 11B. The same configuration portions as those in the first embodiment have the same numerals, and no duplicated description will be made.

The biological information measurement apparatus 1C according to the third embodiment shown in FIG. 11A includes a pinched section 10C and a pinched section accommodating section 15C, which has a pinched section accommodating mechanism that allows at least part of the pinched section 10C to be accommodated in the pinched section accommodating section 15C and the accommodated pinched section 10C to be pulled out by a predetermined amount from the pinched section accommodating section 15C, and the pinched section 10C and the pinched section accommodating section 15C provided at the side surface of a case section (main body section) 2C, which is attached to a user via the band 28.

The pinched section 10C has a strip-like shape readily pinched by the user with fingers. The pinched section 10C has a connected end connected to the case section 2C and a free opposite the connected end, and second electrodes 541C are provided on opposite surfaces of the pinched section 10C on the free end side (see FIG. 11B). The pinched section 10C on the connected end side is so configured that a pinched section accommodating mechanism, for example, a reel mechanism or a slide mechanism allows at least part of the pinched section 10C on the connected end side to be accommodated in the pinched section accommodating section 15C (state shown in FIG. 11A) and the pinched section 10C on the free end side to be pulled out from the pinched section accommodating section 15C (state shown in FIG. 11B).

FIG. 11A shows the state in which no electrocardiogram is measured (second electrodes 541C are not used). In this state, at least part of the pinched section 10C on the connected end side (major part of the pinched section 10C in the present embodiment) is accommodated in the pinched section accommodating section 15C in the case section 2C by using the pinched section accommodating mechanism.

When the second electrodes 541C are used to measure an electrocardiogram, the pinched section 10C is pulled out by a predetermined amount from the pinched section accommodating section 15C in the case section 2C by using the pinched section accommodating mechanism, and a state in which the second electrodes 541C on the pinched section 10C are allowed to be pinched with fingers of the user's right hand is achieved, as shown in FIG. 11B. The second electrodes 541C on the pinched section 10C are then pinched with the thumb and the index finger of the user's right hand, and an electrocardiogram is measured by using the first electrode (not shown), which forms the rear-surface-side electrode on the case section 2C, and the second electrodes 541C.

Since the measurement apparatus 1C according to the present embodiment includes the pinched section accommodating section 15C, which can accommodate at least part of the pinched section 10C on the connected end side when the second electrodes 541C are not used (when no electrocardiogram is measured), the pinched section 10C is advantageously unlikely to bother the user during a period other than acquisition of an electrocardiogram, and the pinched section 10C can be advantageously protected from dirt and stress, as in the second embodiment.

Further, in the measurement apparatus 1C according to the present embodiment, the pinched section 10C, on which the second electrodes 541C are provided, is so arranged that the pinched section 10C can be accommodated in the pinched section accommodating section 15C provided in the case section 2C. Therefore, when it is necessary to exchange the band 28 of the measurement apparatus 1C, the band 28 can be exchanged as required with no consideration of the pinched section 10C.

In the second and third embodiments, in the case where the biological information measurement apparatus is attached to the user's wrist, where the radius and the ulna are present, the pinched section is preferably provided on the radius side. The configuration allows the user to operate the pinched section in a natural posture.

### Fourth Embodiment

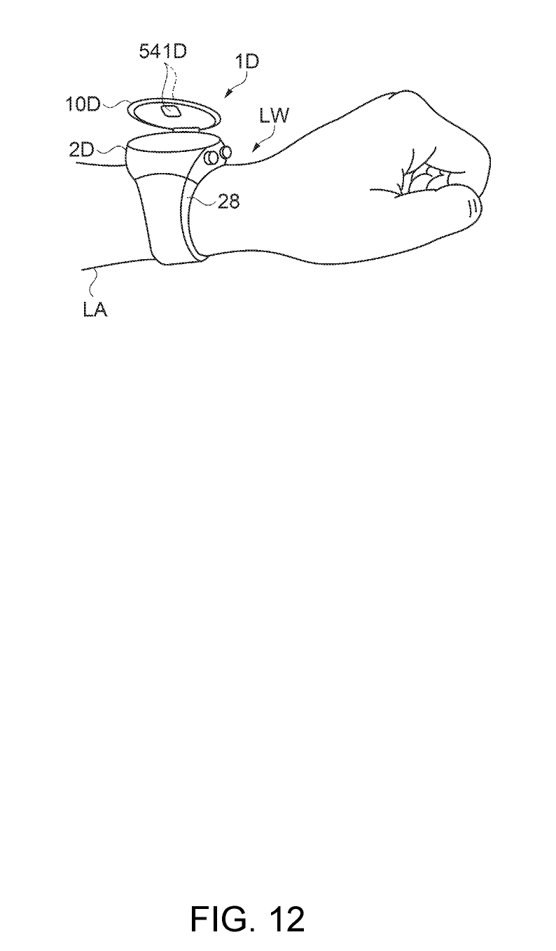

FIG. 12 is a diagrammatic view showing a biological information measurement apparatus according to a fourth embodiment of the invention with the measurement apparatus attached (with measurement apparatus ready for measurement).

A biological information measurement apparatus 1D according to the present embodiment will be described with reference to FIG. 12. The same configuration portions as those in the embodiments described above have the same numerals, and no duplicated description will be made.

In FIG. 12, the measurement apparatus 1D according to the present embodiment includes an upper lid 10D on the front side (the side opposite the rear side that comes into contact with the skin of a user's wrist LW) of a case section (main body section) 2D, which is attached to the user via the band 28, and the upper lid 10D serves as a pinched section so arranged as to be openable and closable via a hinge. The upper lid 10D has a shape readily pinched by the user, and second electrodes 541D are provided on opposite surfaces of a pinched area.

When the second electrodes 541D are used to measure an electrocardiogram, the upper lid 10D as the pinched section is opened via the hinge, and the area (pinched section) of the upper lid 10D where the second electrodes 541D are formed is pinched with the user's fingers (thumb and index finger of right hand, for example), as shown in FIG. 12. In this state, an electrocardiogram is measured by using the first electrode (not shown), which forms the rear-surface-side electrode on the case section 2D, and the second electrodes 541D.

When no electrocardiogram is measured (second electrodes 541D are not used), the upper lid 10D is closed via the hinge.

The measurement apparatus 1D according to the present embodiment, which includes the upper lid 10D, which serves as the pinched section provided with the second electrodes 541D provided on opposite surfaces of the area pinched with the user's fingers, with a minimum increase in size as a wristwatch-shaped biological information measurement apparatus, can provide the same advantageous effects as those provided by the embodiments described above.

The invention is not limited to the embodiments described above, and a variety of changes, improvements, and other modifications can be made to the embodiments described above. Variations will be described below.

### Variation 1

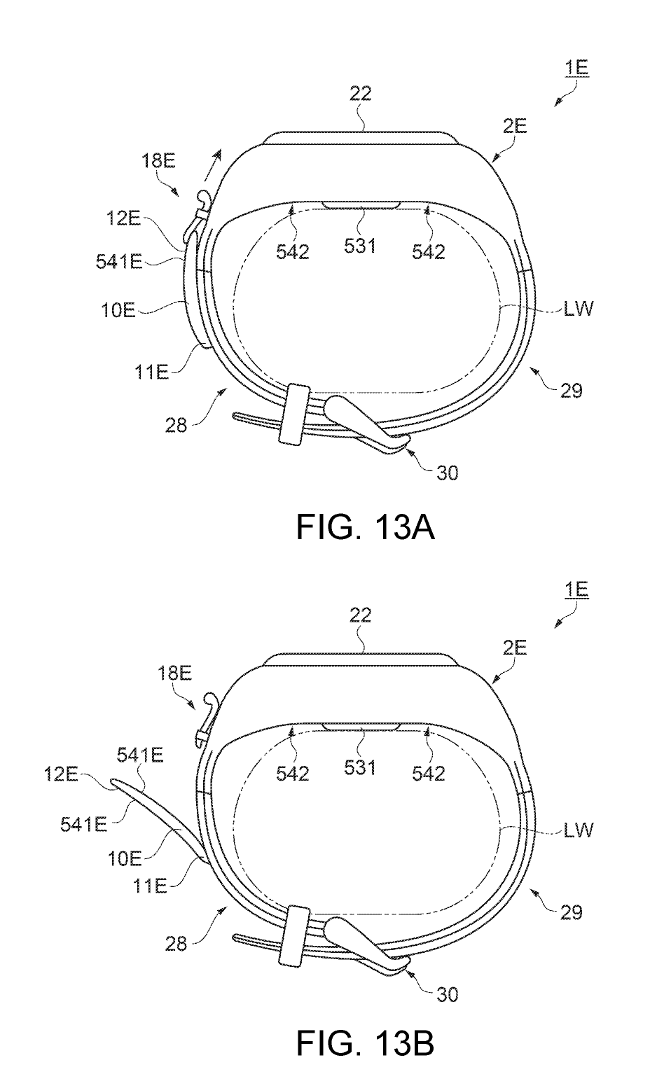

FIGS. 13A and 13B show Variation 1 of the biological information measurement apparatus. FIG. 13A is a diagrammatic view showing a non-measurement state, and FIG. 13B is a diagrammatic view showing a measurement state.

In the second embodiment described above, the description has been made with reference to the configuration in which the band 28 is provided with the pinched section accommodating section 15B having the pinched section accommodating mechanism that allows the pinched section 10B to be accommodated in the pinched section accommodating section 15B and pulled out therefrom and the pinched section 10B is accommodated in the pinched section accommodating section 15B when no electrocardiogram is measured as shown in FIGS. 10A and 10B. The configuration described above is not necessarily employed.

A biological information measurement apparatus 1E according to Variation 1 will be described below. The same configuration portions as those in the embodiments described above have the same numerals, and no duplicated description will be made.

The measurement apparatus 1E according to the present variation is provided with a pinched section 10E having a connected end 11E, which is connected to the band 28, which is one of the bands 28 and 29, via a hinge, and a free end 12E, which is the end opposite the connected end 11E, as shown in FIGS. 13A and 13B. The band 28 is provided with a pinched section locking section 18E including a connector that connects the free end 12E of the pinched section 10E to the connector and a releaser that releases the free end 12E connected to the connector. Second electrodes 541E are provided on opposite surfaces of the pinched section 10E, specifically, in an area thereof that is shifted toward the free end 12E and readily pinched by a user with fingers.

FIG. 13A shows the measurement apparatus 1E in a state in which no electrocardiogram is measured (second electrodes 541E are not used). Specifically, in a state in which the measurement apparatus 1E is attached to a wrist (left wrist) LW of the user's left arm LA, the free end 12E of the pinched section 10E is connected to the band 28 via the connector of the pinched section locking section 18E.

When the second electrodes 541E are used to measure an electrocardiogram, the releaser moves the pinched section locking section 18E in the direction indicated by the arrow in FIG. 13A, and the measurement state shown in FIG. 13B is achieved. Specifically, the pinched section 10E pivots around the hinge located at the connected end 11E of the pinched section 10E, and a state in which the free end 12E separates from the band 28 and the second electrodes 541E are allowed to be pinched with fingers of the user's right hand is achieved. The second electrodes 541E on the pinched section 10E are then pinched with fingers of the user's hand (thumb and index finger of right hand, for example), and an electrocardiogram is measured by using the first electrode (not shown), which forms the rear-surface-side electrode 542 on a case section 2E, and the second electrodes 541E.

According to the measurement apparatus 1E of the present variation, when an electrocardiogram is measured by using the pinched section 10E, on which the second electrodes 541E are provided, the releaser of the pinched section locking section 18E is used to release the connected free end 12E of the pinched section 10E to allow the user to pinch the area of the pinched section 10E where the second electrodes 541E are formed, whereas when no electrocardiogram is measured, the connector of the pinched section locking section 18E can be used to connect the free end 12E of the pinched section 10E to the connector. As a result, the pinched section 10E is advantageously unlikely to bother the user when no electrocardiogram is measured.

### Variation 2

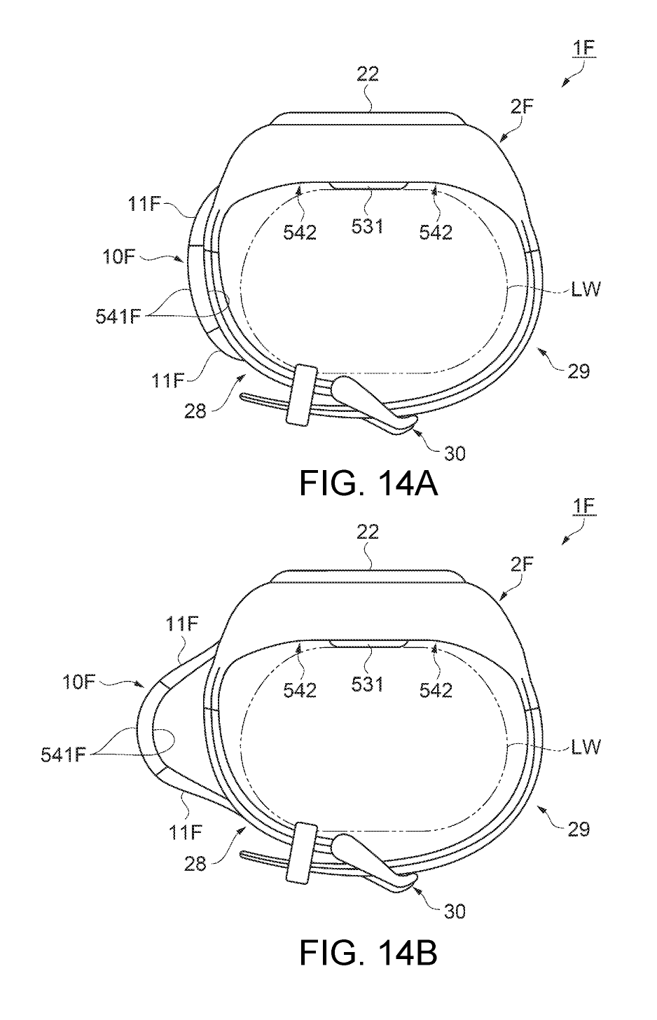

FIGS. 14A and 14B show Variation 2 of the biological information measurement apparatus. FIG. 14A is a diagrammatic view showing a non-measurement state, and FIG. 14B is a diagrammatic view showing a measurement state.

In the embodiments and Variation 1 described above, the description has been made with reference to the configuration including the pinched section having the connected end connected to the band section or the case section and the free end opposite the connected end, but the configuration described above is not necessarily employed.

A biological information measurement apparatus 1F according to Variation 2 will be described below. The same configuration portions as those in the embodiments and Variation 1 described above have the same numerals, and no duplicated description will be made.

The biological information measurement apparatus 1F according to the present variation includes a pinched section 10F having opposite connected ends 11F connected to the band 28, as shown in FIGS. 14A and 14B. In the pinched section 10F, at least a predetermined portion of each of the connected ends 11F, which are opposite ends, is formed of an elastic member. Second electrodes 541F are provided on opposite surfaces of a central predetermined area of the pinched section 10F.

FIG. 14A shows the measurement apparatus 1F in a state in which no electrocardiogram is measured (second electrodes 541F are not used). Specifically, in a state in which the measurement apparatus 1F is attached to a wrist (left wrist) LW of a user's left arm LA, the pinched section 10F is so arranged as to be in intimate contact with the band 28 or separate therefrom with a small gap.

When the second electrodes 541F are used to measure an electrocardiogram, the user's finger (index finger of right hand, for example) is inserted into the gap between the pinched section 10F and the band 28, and the pinched section 10F is pulled with the aid of elasticity of at least the predetermined portions of the opposite connected ends 11F, so that a gap between the band 28 and the pinched section 10F is so provided as to be wide enough for the user to pinch the area of the pinched section 10F where the second electrodes 541F are formed. The second electrodes 541F on the pinched section 10F are then pinched with fingers of the user's hand (thumb and index finger of right hand, for example), and an electrocardiogram is measured by using the first electrode (not shown), which forms the rear-surface-side electrode 542 on the case section 2F, and the second electrodes 541F.

According to the measurement apparatus 1F of the present variation, not only is the pinched section 10F advantageously unlikely to bother the user when no electrocardiogram is measured, as in the embodiments and Variation 1 described above, but also the pinched section 10F, which achieves stable contact between the second electrodes 541F and the user's body, can be advantageously arranged by using a simpler configuration with no complicated mechanism than in the embodiments and Variation 1 described above.

### Fifth Embodiment

A fifth embodiment of the invention will next be described.

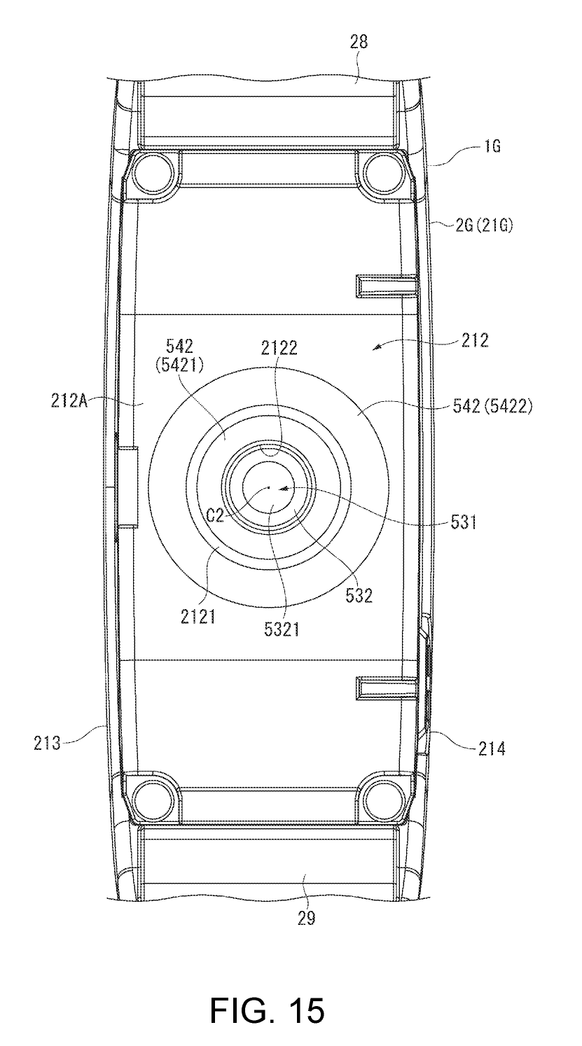

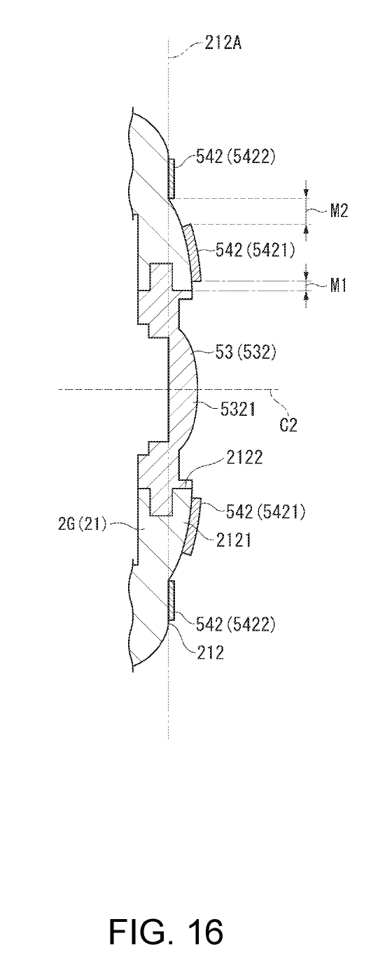

FIG. 15 is a rear view showing a biological information measurement apparatus according to a fifth embodiment of the invention. FIG. 16 is a cross-sectional view showing a main body section and a light transmissive member in the fifth embodiment.

The biological information measurement apparatus according to the present embodiment has the same configuration as that of each of the biological information measurement apparatus 1A to 1F described above but differs therefrom in that the rear-surface-side electrode is arranged differently. In the following description, portions that are the same or roughly the same as those having been already described have the same reference characters and will not be described.

FIG. 15 is a rear view showing a biological information measurement apparatus 1G according to the present embodiment. FIG. 16 is a cross-sectional view showing a portion of a case section 2G (main body section 21G) on the side facing the rear surface 212 and a light transmissive member 532, which forms the pulse wave sensor 531. FIG. 16 is a cross-sectional view taken along a line that connects the front surface 211 to the rear surface 212.

The biological information measurement apparatus 1G according to the present embodiment has the same configuration as that of the biological information measurement apparatus 1A described above except the configuration of the rear surface 212 and the arrangement of the rear-surface-side electrode 542.

In the measurement apparatus 1G, a protrusion 2121 is formed on the rear surface 212 of the main body section 21G, which forms the case section 2G, as shown in FIG. 15. The protrusion 2121 is formed in a gentle convex curved shape in which the amount of protrusion from a reference plane 212A of the rear surface 212 (flat plane that connects corners of rear surface 212 to one another) increases with distance from the outer edge of the rear surface 212 toward the center C2, as shown in FIG. 16. That is, the protrusion 2121 is so shaped that the position at the center C2 protrudes by a greater amount from the reference plane 212A than the positions on the outer edge of the rear surface 212.

A detection window 2122, which is a circular opening, is formed at the center of the protrusion 2121, as shown in FIGS. 15 and 16. The light transmissive member 532, which forms the pulse wave detector 53 described above, is fit into the detection window 2122, and the light transmissive member 532 covers the light emitting device and the light receiving device (not shown) of the pulse wave sensor 531 provided in the main body section 21G. That is, the protrusion 2121 also functions as a light blocker that prevents light from passing through portions other than the detection window 2122 and impinging on the light receiving device of the pulse wave sensor 531.

A raised section 5321, which is raised in an arcuate shape, is formed roughly at the center of the light transmissive member 532. With reference to the reference place 212A described above, the position of the raised section 5321 is higher than the position of the highest portion of the protrusion 2121, the end thereof facing the center C2. That is, the vertex of the raised section 5321 is separate from the reference plane 212A by a greater amount than the protrusion 2121.

The electrodes that form the rear-surface-side electrode 542 arranged on the rear surface 212 are so configured that the first electrode 5421 is arranged in an annular shape on the protrusion 2121 described above and in a portion shifted toward the detection window 2122, and the indifferent electrode 5422 is arranged in an annular shape in an outer portion of the protrusion 2121.

The first electrode 5421 is arranged on the protrusion 2121 and in a position shifted from the outer edge of the protrusion 2121 toward the center C2. In other words, the first electrode 5421 is arranged on the protrusion 2121 in such a way that a dimension M1 between the first electrode 5421 and the edge of the detection window 2122 is smaller than dimension M2 between the first electrode 5421 and the outer edge of the protrusion 2121 when viewed from a position facing (directly facing) the rear surface 212. The position of the first electrode 5421 from the reference plane 212A is higher than the position of the raised section 5321 described above. In detail, the first electrode 5421 is arranged is a position most remote from the reference plane 212A among the portions present on the rear surface 212.

The measurement apparatus 1G according to the present embodiment described above can provide the following advantageous effects as well as those provided by the measurement apparatus 1A to 1F described above.

The first electrode 5421 of the rear-surface-side electrode 542 is arranged on the protrusion 2121 described above. Therefore, when the measurement apparatus 1G is attached to the attachment site described above so that the raised section 5321 comes into intimate contact with the attachment site, the first electrode 5421 is allowed to come into intimate contact with the attachment site. An electrocardiogram can therefore be detected with precision.

In the measurement apparatus 1G described above, the rear-surface-side electrode 542 has the first electrode 5421 and the indifferent electrode 5422. The rear-surface-side electrode 542 may, however, have a configuration in which only the first electrode 5421 is provided or a configuration in which in addition to the first electrode 5421 and the indifferent electrode 5422, another electrode is provided.

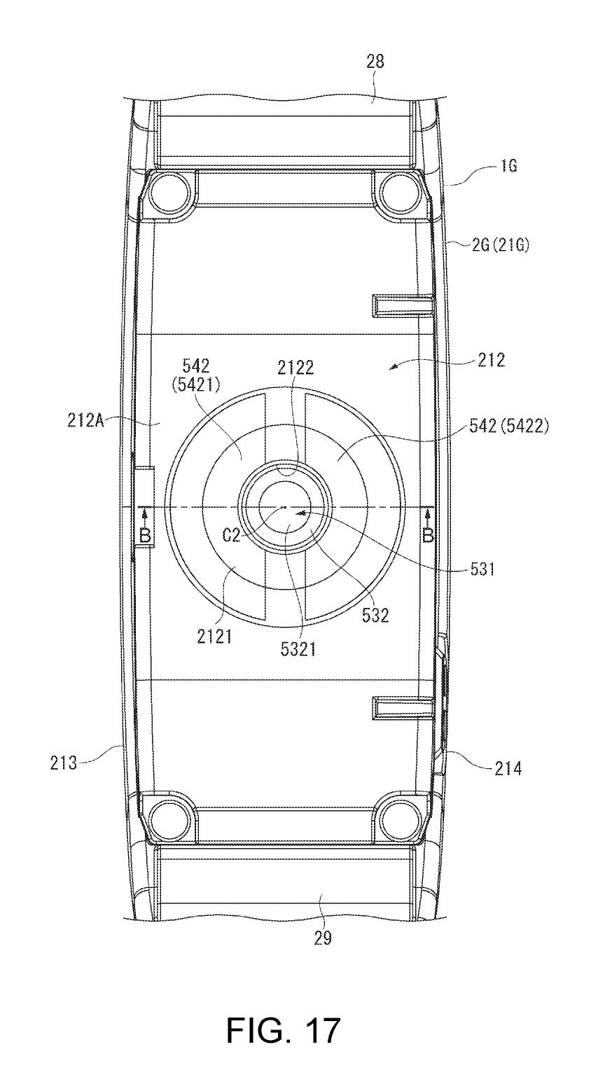

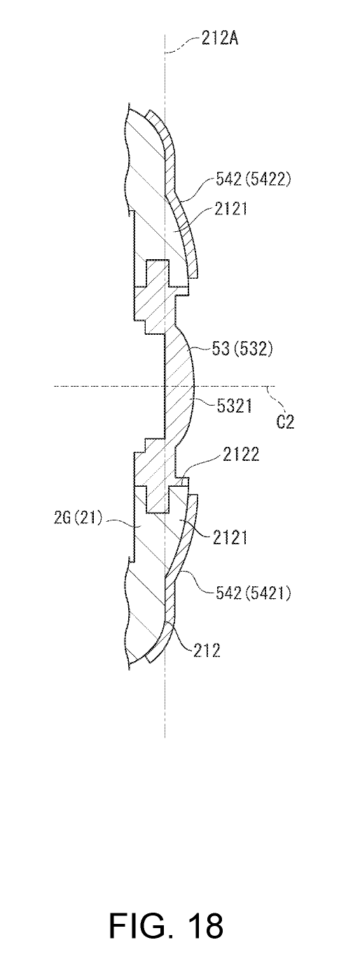

Further, the electrodes that form the rear-surface-side electrode 542 each do not necessarily has an annular shape and only need to be divided into a plurality of electrodes. FIGS. 17 and 18 show another aspect of the arrangement and shape of each of the electrodes that form the rear-surface-side electrode 542. FIG. 17 is a rear view, and FIG. 18 is a cross-sectional view taken along the line B-B in FIG. 17. The rear-surface-side electrode 542 of the measurement apparatus 1G shown in FIG. 17 is so configured that the first electrode 5421 and the indifferent electrode 5422 are arranged side by side with a gap therebetween. In FIG. 17, the first electrode 5421 is arranged on the rear surface 212 on the left in the plane of view, and the indifferent electrode 5422 is arranged on the rear surface 212 on the right in the plane of view. Part of the first electrode 5421 and the indifferent electrode 5422 is arranged on the protrusion 2121, which protrudes toward a user's body (skin), on the rear surface 212 (see FIG. 18).

The arrangement and shape of each of the electrodes that form the rear-surface-side electrode 542 described above allow both the first electrode 5421 and the indifferent electrode 5422 to be arranged on the protrusion 2121, which protrudes toward the user's body (skin), on the rear surface 212 and further so arranged as to spread over a relatively wide area of the protrusion 2121, whereby the contact between the rear-surface-side electrode 542 and the user's skin is satisfactorily maintained for more accurate electrocardiogram acquisition.

The configuration of the measurement apparatus 1G described above may be changed to a configuration including a case section having a roughly rectangular main body section when viewed from the rear side or a configuration including a case section having a roughly circular main body section when viewed from the rear side, and the configuration of the rear-surface-side electrode described above may be applied to the rear surface of any of the main body sections described above.

The variations of the configuration described above are also applicable to the first to fourth embodiments and variations thereof as well as the measurement apparatus 1G according to the present embodiment.

The embodiments of the invention made by the inventor have been specifically described above, but the invention is not limited to the embodiments described above, and a variety of changes can be made thereto to the extent that the changes do not depart from the substance of the invention.

For example, in the measurement apparatus 1A to 1F according to the embodiments described above, the shapes of the pinched sections 10A to 10F, on which the second electrodes 541A to 541F are provided, are not limited to the shapes shown in the drawings and may each be any shape that allows the state in which the pinched sections are pinched with the user's fingers to be readily maintained.

Further, the electrodes that keep in contact with a user's body when the measurement apparatus is attached thereto (the first electrode 5421 and the indifferent electrode 5422 as the rear-surface-side electrode 542 in the embodiments described above) are not necessarily arranged on the rear surface of the case section (main body section) and only need to be arranged on a surface that comes into contact with the user's skin in the state in which the measurement apparatus is attached thereto. For example, the electrodes may instead be provided on the inner side of any of the bands 28 and 29 (the side that comes into contact with the user's skin in the attached state). In this case, the electrodes can be connected to the main body section by providing a signal line (electric wire) on the inner or outer side of the band 28 or 29 along the band 28 or 29.

In the embodiments described above, the first electrode 5421 and the indifferent electrode 5422, which form the rear-surface-side electrode 542, are concentrically disposed around the center C2. The invention is, however, not necessarily configured this way. That is, the arrangement of the first electrode 5421 and the indifferent electrode 5422 may be changed as appropriate. For example, the first electrode 5421 and the indifferent electrode 5422 may be arranged side by side on the rear surface 212.

In each of the embodiments described above, the biological information detector 52 has not only the electrocardiogram measurement section 54 but also the pulse wave detector 53, which detects the user's pulse wave. The invention is, however, not necessarily configured this way. That is, the pulse wave detector 53 may be omitted, and the biological information detector 52 may having a configuration further including a detector that detects other types of biological information (blood pressure, blood sugar level, body temperature, sweat rate, and bioelectrical impedance, for example). Further, the body motion information detector 51 may be omitted.

In the embodiments and variations thereof described above, the second electrodes 541A to 541F and the pinched sections 10A to 10F, on which the second electrodes 541A to 514F are provided, are used only for measurement of a user's electrocardiogram. The invention is, however, not necessarily configured this way. For example, the electrodes may be configured to be usable as an operational pinched section that form the operation section 4 described above when no electrocardiogram is measured.

In each of the embodiments described above, the case sections 2A to 2F are attached to a user's body via the pair of bands 28 and 29 as the attachment member. The invention is, however, not necessarily configured this way. That is, the attachment member may be configured in any manner as long as the attachment member allows the measurement apparatus 1A to 1F to be attached to the user's body. Further, the bands 28 and 29 may be integrated with the case sections 2A to 2F, as described above.

In each of the embodiments described above, each of the measurement apparatus 1A to 1F is a wristwatch-shaped wearable apparatus attachable to a user's left wrist LW. The invention is, however, not necessarily configured this way. The measurement apparatus may have a roughly box-like shape or any other shape. Further, the measurement apparatus 1A to 1F are not necessarily attached to the left wrist LW and may instead be attached to a right wrist or any other body part.

What is claimed is:

1. A biological information measurement apparatus comprising:
a case section including a biological information detector that detects biological information on a user;
a band section that attaches the case section to the user;
a first electrode provided on a contact surface of the case section or the band section that comes into contact with the user's body;
a pinched section provided as part of the case section or the band section and pinched by the user with fingers;
a second electrode provided on the pinched section; and
an electrocardiogram detector that uses the first electrode and the second electrode to detect the user's electrocardiogram.

2. The biological information measurement apparatus according to claim 1,
wherein the second electrode is provided on each principal surface of the pinched section.

3. The biological information measurement apparatus according to claim 1,
wherein the band section has one end connected to the case section and another end that is a free end, and
the pinched section is arranged at the other end of the band section.

4. The biological information measurement apparatus according to claim 1,
wherein the pinched section has a connected end connected to the case section or the band section and a free end capable of releasing connection thereof to the case section or the band section.

5. The biological information measurement apparatus according to claim 1, further comprising
a pinched section accommodating section having an accommodating mechanism capable of adjusting a length of the pinched section.

6. The biological information measurement apparatus according to claim 5,
wherein the accommodating mechanism is capable of adjusting the length of the pinched section in such away that the length of the pinched section in a case where the second electrode is used is greater than the length of the pinched section in a case where the second electrode is not used.

7. The biological information measurement apparatus according to claim 5,
wherein the accommodating mechanism is formed of a reel mechanism or a slide mechanism.

8. The biological information measurement apparatus according to claim 1,
wherein the pinched section is provided as part of the band section along a direction in which the band section extends and a direction in which the pinched section extends.

9. The biological information measurement apparatus according to claim 1,
wherein a third electrode insulated from the first electrode is provided on the surface on which the first electrode is arranged.

10. The biological information measurement apparatus according to claim 9,
wherein the third electrode is an indifferent electrode.

11. The biological information measurement apparatus according to claim 1, further comprising
a pulse wave detector that is provided on the contact surface of the case section that comes into contact with the user's body and detects the user's pulse wave.

12. The biological information measurement apparatus according to claim 11,
wherein in a cross-sectional view viewed along a direction parallel to the contact surface of the case section that comes into contact with the user's body, the pulse wave detector is provided on a protrusion that protrudes from the case section toward the user's skin, and at least part of the first electrode is provided on the protrusion.

13. The biological information measurement apparatus according to claim 1, further comprising
a detection window which is provided through the surface on which the first electrode is arranged and through which the user's pulse wave is detected.

14. The biological information measurement apparatus according to claim 13,
wherein in a side view viewed along a direction parallel to the contact surface of the case section that comes into contact with the user's body, the case section has a protrusion that is adjacent to the detection window and protrudes from the case section toward the user's skin, and at least part of the first electrode is provided on the protrusion.

15. The biological information measurement apparatus according to claim 1,
wherein the second electrode has a recess.
